(19) United States
(12) Patent Application Publication
(10) Pub. No.: US 2008/0268001 A1
(43) Pub. Date: Oct. 30, 2008

(54) ORAL CARE COMPOSITION TO REDUCE OR ELIMINATE DENTAL SENSITIVITY

(76) Inventors: Lynette Zaidel, Cranford, NJ (US); Suman K. Chopra, Monroe, NJ (US); Michael Prencipe, Princeton Junction, NJ (US); Qin Wang, Monmouth Junction, NJ (US); Constantina Christopoulou, Somerset, NJ (US)

Correspondence Address: COLGATE-PALMOLIVE COMPANY, 909 RIVER ROAD, PISCATAWAY, NJ, 08855, US

(21) Appl. No.: 11/742,039

(22) Filed: Apr. 30, 2007

(51) Int. Cl. A61K 8/02; A61Q 11/00

(52) U.S. Cl. 424/401; 424/56

(57) ABSTRACT

The invention includes an oral care composition that reduces and/or eliminates the perception of tooth sensitivity. The composition includes an adherent material and silica particles having an average particle size of about 8 microns or less. The silica particles are present in the composition in an amount of about 5% by weight. Also included within the scope of the invention are related methods, such as methods of occluding a dentin tubule.

### BACKGROUND OF THE INVENTION

Dentin is a portion of the tooth internal to the enamel and cementum that has a radially striated appearance owing to a large number of fine canals or tubules known as the dentinal tubules. Tubules run from the pulp cavity to the periphery of the dentin and are generally about two microns in diameter at their base and somewhat narrower at their periphery. Tubules are not usually exposed to the environment in the oral cavity, as they are usually covered by enamel or cementum. The cementum in turn is often covered by the gums.

It is commonly understood that partially or fully exposed tubules can lead to tooth sensitivity, an irritating and painful condition. In this theory, recession of the gum line exposes cementum to erosion. The eroded cementum in turn exposes the hollow dentinal tubules. The exposed tubules cause nerves within the tooth to be affected excessively by external oral stimuli because material and energy transfer between the exterior and interior of the tooth is accelerated through the tubules. Common environmental stimuli, such as heat, cold, chemicals and physical and mechanical pressure or stimuli, such as brushing, are able to irritate the nerve through the open dentin tubules and thereby create pain. The pain of sensitive teeth appears to result from these stimuli, which apparently cause fluid movements in the dentinal tubules that activate pulpal nerve endings.

Conventionally, two approaches have been taken to treat or ameliorate tooth sensitivity. Under one approach, the chemical environment proximal to the nerve is altered by application of various agents, such that the nerve is not stimulated, or not stimulated as greatly. Known agents useful in this chemical approach, including potassium salts (such as potassium nitrate, potassium bicarbonate, potassium chloride) and strontium, zinc salts, and chloride salts.

The second approach involves the mechanical shield of the nerve by, e.g., blocking of the dentinal tubules wholly or partially with “tubule blocking agents.” Agents that have been disclosed in the prior art include, e.g., cationic alumina, clays, water-soluble or water-swellable polyelectrolytes, maleic acid copolymers and polyethylene particles.

However, both the chemical and the mechanical approaches, because they require the incorporation of one or more additional materials to the dentifrice, may result in formulation difficulties, either technical or related to increased costs. For this reason there is a need in the art for a dentifrice that, upon use, prevents or reduces tooth sensitivity, yet is not associated with significant processing or formulation disadvantages.

### BRIEF SUMMARY OF THE INVENTION

The invention includes an oral care composition that reduces and/or eliminates the perception of tooth sensitivity. The composition includes an adherent material and silica particles having an average particle size of about no greater than about the diameter of a dentin tubule, or alternatively about 8 microns or less. The silica particles are present in the composition in an amount of about 5% by weight, or greater. In an alternative, the silica particles may be present in an amount of about 5% to about 25% by weight. The compositions described provide a reduction in fluid flow of no greater than about 25% of the fluid flow value of etched dentin.

Also included within the scope of the invention are related methods, such as methods of occluding a dentin tubule.

### BRIEF DESCRIPTION OF THE DRAWINGS

FIG. 1 shows a comparison of the occlusion incidence of composition A versus composition B in an acid-treated mammalian tooth substrate.

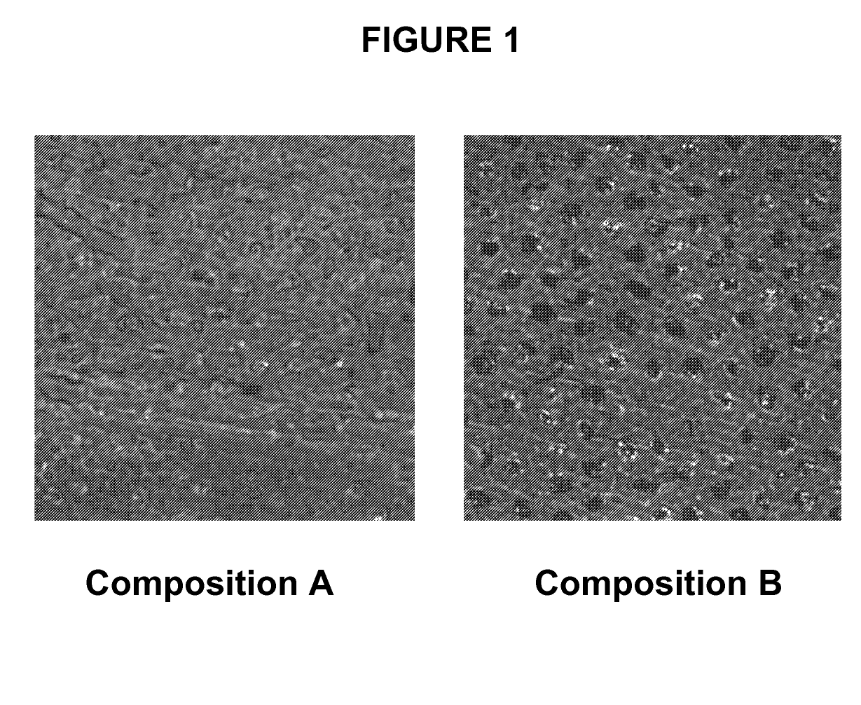

### DETAILED DESCRIPTION OF THE INVENTION

The invention described herein includes an oral care composition that contains at least (a) an adherent material and (b) a silica particle. The silica particle may have an average particle size of about no greater than a dentin tubule, or alternatively it may have an average particle size of 8 microns or less. The silica particles may be present in an amount of about 5% by weight or greater. The compositions may contain additional therapeutic and non-therapeutic components, and may also be utilized in the practice of various methods, all of which are included within the scope of the invention. The composition and methods within the scope of the invention may be useful in, for example, reducing or eliminating tooth sensitivity of a mammal, improving/maintaining systemic health, and/or occluding dentin tubules.

The oral compositions of the invention include an adherent material. The adherent material may be any known or to be developed in the art that attaches to the surface of a mammalian tooth and/or to the heterogenous biofilm which also may be present on a tooth's surface. Attachment may occur by any means, such as ionic interaction, van der Waals forces, hydrophobic-hydrophilic interactions, etc. The adherent material may be, for example, chitosan, chitin, a gum or a marine colloid. Other contemplated adherent materials include any homopolymers or copolymers (hereinafter referred to collectively as a “polymer”) that adhere to the surface of a tooth. Such polymers may include silicone polymers, polymers having monomers of polyvinyl phosphonic acid, poly(1-phosphonopropene), sulfonic acid, poly(beta styrene phosphonic acid), alpha styrene phosphonic acid, synthetic anionic polymeric polycarboxylate, maleic anhydride, maleic acid, and methyl vinyl ether. Polymers of any molecular weight may be used, including, for example molecular weights about 1,000 to about 5,000 (number average). In various embodiments, one may use a copolymer of methyl vinyl ether and maleic anhydride, in for example, a monomer ratio of about 1:4 to about 4:1. Other polymers that may be used as adherent materials include those recited in U.S. Pat. Nos. 4,521,551; 4,485,090; 4,138,477; 4,138,914; and 3,956,480, the contents of each of which are incorporated herein by reference.

The oral compositions within the scope of the invention also include silica particles that have an average particle size that is no greater than about the average diameter of a mammalian dentin tubule, such that one or more particles is/are capable of becoming lodged within the tubule, thereby effecting a reduction or elimination of perceived tooth sensitivity. Suitable particles may have, for example, an average particle size of about 8 microns or less, alternatively, about 3 to about 4 microns, or about 5 to about 7 microns. The silica particles may be initially present in the composition having the desired particle size, or may be initially present in the composition at a larger size, so long as the structure of the particles is such that it fractures or breaks into the desired particle size upon application of mechanical force by, e.g., a toothbrush, when brushing.

The silica particle may be prepared by any means known or to be developed in the art, and may be surface modified, if desired, to increase the capacity of the particle to adhere to a tooth surface. Examples may be found in, e.g., U.S. patent application Ser. No. 11/271,306, the contents of which are incorporated herein by reference. The silica particle is present in the composition in an amount of about 5% or greater by weight of the total composition. Alternatively, the silica particle may be present in an amount of about 5%, about 10%, about 15%, about 20% or about 25% by weight.

Any type of silica may be used, such as precipitated silicas or silica gels. Preferred are commercially available silicas such as INEOS AC43 or MD929, available from Ineos Silicas, Warrington, United Kingdom, and a silica sold under the name XWA300, available from Grace Davison, 7500 Grace Drive, Columbia, Md. 21044, United States of America.

The oral care compositions described herein may be formulated into any delivery form that permits contact of the adherent material and the silica particles, to the tooth surface. For example, the compositions may be formulated into a mouth rinse, a paste, a gel, a lozenge (dissolvable or chewable), a spray, a gum, and a film (wholly or partially dissolvable, or indissoluble). The composition may contain any conventional excipients or carriers, although these will vary depending on the dosage form or means of dosage selected. Excipents or carriers can include, for example, humectants, colorants, flavorants, glycerin, sorbitol, xylitol, and/or propylene glycol, water or other solvents, gum bases, thickening agents, surfactants, carrageenan (rich moss), xanthan gum and sodium carboxymethyl cellulose, starch, polyvinyl pyrrolidone, hydroxyethyl propyl cellulose, hydroxybutyl methyl cellulose, hydroxypropyl methyl cellulose, and hydroxyl ethyl cellulose and amorphous silicas.

Surfactants may be included, if desired. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of the last mentioned amides include N-lauryl sarcosine, and the sodium, potassium and ethanolamine salts of N-lauryl, N-myristoyl, or N-palmitoyl sarcosine. Others include, for example, nonanionic polyoxyethylene surfactants, such as Polyoxamer 407, Steareth 30, Polysorbate 20, and castor oil; and amphoteric surfactants, such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from about 12 to about 20 carbon atoms), which condensation products (ethoxamers) contain hydrophilic polyoxyethylene moieties, such as condensation products of poly (ethylene oxide) with fatty acids, fatty alcohols, fatty amides and other fatty moieties, and with propylene oxide and polypropylene oxides.

In an embodiment, the oral composition includes a surfactant system that is sodium laurel sulfate (SLS) and tauranol. If desired, the SLS and tauranol may be present in a ratio of about 1:5 to about 1:3.

Dentin that is treated with the combination of the invention produce a fluid flow rate of no greater than about 25%, about 20%, about 15% or about 10%, as determined by the Dentin Conductance Procedure.

Dentin Conductance Procedure: Extracted human molars are cut at the crown and roots using a diamond saw. The pulp is removed and the resulting dentin segment is stably mounted, such as onto an acrylic block. Tubing is connected from a hole in the acrylic block mounting just below the pulp chamber. The dentin segment is connected to an apparatus that measures the rate of fluid flow (hydraulic conductance).

The top surface of the dentin is etched with citric acid. The fluid flow rate across the etched dentin is measured. The dentin surface is then treated with the oral composition of the invention and the fluid flow rate is measured again. See, Zhang et al., The Effects of pain free desensitizer or dentine permeability and tubule occlusion over time, in vitro. J. Clin. Periodontol., 1998, the contents of which are incorporated herein by reference.

The oral care composition may include any other therapeutic, cosmetic, and/or aesthetic materials as may be desired. Examples include desensitizing agents, a nitrate salt, an arginine ester, a bicarbonate salt, potassium nitrate, and an arginine-bicarbonate-phytate complex, a chemical whitening agent (such as a peroxide releasing compound), an opaque whitening agent (such as hydroxyapetite) and an anticalculus agent. Other options for inclusion in the oral care composition of the invention include triclosan; stannous ion agents; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agents; copper ion agents; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, an enzyme, a Camellia extract, a flavonoid, a flavan, halogenated diphenyl ether, creatine, and propolis.

The oral care composition of the invention may be prepared by any means known in the art. For example, preparation methods for dentifrices are well known, for example, as described in U.S. Pat. Nos. 3,966,863; 3,980,767; 4,328,205; and 4,358,437, the contents of which are incorporated herein by reference. In general, any humectant (e.g., glycerin, sorbitol, propylene glycol, and/or polyethylene glycol) is dispersed in water in a conventional mixer under agitation. Into that dispersion are added the thickeners, such as carboxyl methyl cellulose (CMC), carrageenan, or xanthan gum; any anionic polycarboxylate; any salts, such as sodium fluoride anticaries agents; and any sweeteners.

The resultant mixture is agitated until a homogeneous gel phase is formed. Into the gel phase are added any pigments utilized, such as TiO₂, and additionally any acid or base required to adjust the pH of the composition. These ingredients are mixed until a homogeneous phase is obtained.

The mixture is then transferred to a high speed/vacuum mixer, wherein the surfactant ingredients are added to the mixture. The silicas utilized are added subsequently. Any water insoluble agents, such as triclosan, are solubilized in the flavor oils to be included in the dentifrice, and that solution is added along with the surfactants to the mixture, which is then mixed at high speed for about 5 to about 30 minutes, under a vacuum of about 20 to about 50 mm of Hg. The resultant product is a homogeneous, semi-solid, extrudable paste or gel product.

The invention also includes within its scope several related methods. For example, the invention includes within its scope methods of reducing dental sensitivity and methods of occluding a dentin tubule of a mammalian tooth.

Each of these methods includes the steps of applying any of the compositions described above to the tooth surface. Application may be carried out by any method, so long as the adherent material and the silica particles are placed in contact with the tooth surface. Application may be accomplished by brushing, flossing, irrigating, wiping, rinsing (lavage of oral cavity), foam/gel and in-tray application, masticating, spraying, painting, etc., or applied by film or strip.

Alternatively, the invention includes methods to increase or maintain the systemic health of a mammal by applying a composite to an oral surface (both hard and soft tissues of the oral cavity). The composition for use in this method may be any described above, provided that it contains at least one of triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and propolis. The application may be at least once a day, although up to five times per day may be preferred, and may be carried out over a duration of time, e.g., one week, up to one year, up to three years or for a lifetime.

### EXAMPLE 1

Two compositions paste-form was prepared using the materials and amounts set out in Table I and the process described below. Composition A represents a composition within the scope of the invention and composition B is a control composition that does not contain the specified silica particle.

TABLE I

| | A | B |
|---|---|---|
| Water | 15.607 | 15.607 |
| Saccharin | 0.3 | 0.3 |
| NaF | 0.243 | 0.243 |
| Glycerin | 20 | 20 |
| Propylene Glycol | 0.5 | 0.5 |
| Carboxy methyl cellulose (CMC) | 1.1 | 1.1 |
| Iota Carrageenan | 0.4 | 0.4 |
| TiO2 | 0.5 | 0.5 |
| Sorbitol | 20.85 | 20.85 |
| PMV/MA Copolymer | 15 | 15 |
| NaOH | 1.2 | 1.2 |
| Abrasive silicas | 15 | 20 |
| Ineos AC43 | 5 | — |
| Flavor | 1 | 1 |
| triclosan | 0.3 | 0.3 |
| Sodium laureth sulfate | 1.5 | 1.5 |
| Total | 100 | 100 |

Sodium saccharin and sodium fluoride was dissolved in water. Triclosan was dissolved in flavor.

Glycerin and propylene glycol were mixed together. Sodium CMC and iota carragenan was dispersed. Titanium dioxide was added to the mixture. This was followed by the addition of sorbitol. To this sodium saccharin and sodium fluoride in water was added and it was mixed for 15 minutes at 49° C. Then the PMV/MA copolymer and sodium hydroxide (50%) were added at 49° C. (5 minutes mixing). The whole mixture was dropped into a mixer and mixed. Subsequently the abrasive silicas and the Ineos AC43 silica particles were added at high speed under full vacuum.

Premix flavor and triclosan and sodium sulphate powder were added. It was mixed for 10 minutes at medium speed under full vacuum. The vacuum was released and the whole batch was inspected for uniformity.

Fluid flow across dentin samples using each composition (A & B) was measured using the procedure described above.

1. An oral care composition comprising:
a. an adherent material;
b. silica particles having an average particle size of about 8 microns or less, wherein the silica particles are present in the composition in an amount of about 5% by weight or greater, and the composition provides a fluid flow rate of no greater than about 25% of the fluid flow rate of untreated dentin.

2. The composition of claim 1, wherein the adherent material is a polymer.

3. The composition of claim 1, wherein the polymer has a number average molecular weight of about 1,000 to about 5,000.

4. The composition of claim 1, wherein the adherent material is selected from polymers of polyvinyl phosphonic acid, poly(1-phosphonopropene), sulfonic acid, poly(beta styrene phosphonic acid), alpha styrene phosphonic acid, synthetic anionic polymeric polycarboxylate, maleic anhydride, maleic acid, and methyl vinyl ether.

5. The composition of claim 1, wherein the adherent molecule is a polymer of methyl vinyl ether and maleic anhydride.

6. The composition of claim 1, wherein the silica particle has an average particle size of about 3 to about 5 microns.

7. The composition of claim 1, wherein the silica particle has an average particle size of less than about 7 microns.

8. The composition of claim 1, wherein the composition is formulated into a form selected from a rinse, a paste, a gel, a gum, a dissolvable lozenge, and a film.

9. The composition of claim 8, wherein the film is a dissolvable film.

10. The composition of claim 1 further comprising a desensitizing agent.

11. The composition of claim 1 further comprising a desensitizing agent selected from a nitrate salt, an arginine ester, a bicarbonate salt, potassium nitrate, and an arginine-bicarbonate-phytate complex.

12. The composition of claim 1 further comprising an antibacterial agent.

13. The composition of claim 1 further comprising an agent selected from a chemical whitening agent, an opaque whitening agent and an anticalculus agent.

14. The composition of claim 1 further comprising triclosan.

15. The composition of claim 1 further comprising a surfactant system that comprises sodium lauryl sulfate and tauranol.

16. The composition of claim 1 further comprising a surfactant system that consists essentially of sodium lauryl sulfate and tauranol in a ratio of about 1:5 to about 1:3.

17. The composition of claim 1 further comprising an agent selected from a stannous ion agent; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and propolis.

18. A method of reducing dental sensitivity comprising applying to the surface of a mammalian tooth a composition comprising an adherent material and a silica particle having an average particle size of about b [microns or less, wherein the silica particles are present in the composition in an amount of about 5% by weight or greater.

19. The method of claim 18, wherein the adherent material is selected from polymers of polyvinyl phosphonic acid, poly(1-phosphonopropene), sulfonic acid, poly(beta styrene phosphonic acid), alpha styrene phosphonic acid, synthetic anionic polymeric polycarboxylate, maleic anhydride, maleic acid, and methyl vinyl ether.

20. The method of claim 18, wherein the silica particle has an average particle size of less than about 7 microns.

21. The method claim 18, wherein the application of the composition to the tooth surface is carried out by one of rinsing, brushing, wiping, masticating, and spraying.

22. A method of maintaining or increasing the systemic health of a mammal comprising applying a composition to an oral surface of a mammal at least once a day for a duration of time, wherein the composition comprises an adherent polymer, a silica particle having an average particle size of about 8 microns or less, wherein the silica particles are present in the composition in an amount of about 5% by weight or greater, and an agent selected from triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-etlhylpyridinium chloride (TDEPC); octenidine; delmopinol; octapinol; nisin; zinc ion agent; copper ion agent; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and propolis.

23. The method of claim 22, wherein the duration of time is about 1 month to about 1 year.

24. The method of claim 19, wherein the silica particle of the composition has an average particle size of less than about 7 microns.

25. A method of occluding a dentin tubule within the surface of a mammalian tooth comprising applying to the tooth surface a composition comprising an adherent material and a silica particle having an average particle size of about no greater than a dentin particle.
